(19) United States
(12) Patent Application Publication
(10) Pub. No.: US 2011/0111061 A1
(43) Pub. Date: May 12, 2011

(54) Compositions and Methods for the Treatment of Skeletal Metastatic Lesions and Fractures

(76) Inventors: John A. Handal, Bryn Mawr, PA (US); Solomon P. Samuel, Philadelphia, PA (US)

(21) Appl. No.: 12/950,216

(22) Filed: Nov. 19, 2010

Related U.S. Application Data
(63) Continuation-in-part of application No. PCT/US09/44664, filed on May 20, 2009.
(60) Provisional application No. 61/054,638, filed on May 20, 2008.

(51) Int. Cl. A61K 33/00; A61K 47/30; A61K 47/32; A61K 47/02; A61K 31/4985; A61K 31/704; A61P 35/00; A61P 19/08

(52) U.S. Cl. 424/715; 514/772.3; 514/772.4; 514/769; 514/249; 514/34

(57) ABSTRACT

Compositions and methods for the treatment of fracture and metastatic bone cancer are disclosed.

This application is a continuation-in-part of PCT/US2009/044664, filed on May 20, 2009, which claims priority under 35 U.S.C. §119(e) to U.S. Provisional Patent Application No. 61/054,638, filed on May 20, 2008. The foregoing applications are incorporated by reference herein.

### FIELD OF THE INVENTION

The present invention relates to the fields of medicine, cancer treatment, osteoconduction, osteoinduction and osteogenesis. More specifically, the invention provides compositions and methods to facilitate bone healing or restoration in response to fracture and/or malignancy.

### BACKGROUND OF THE INVENTION

Several publications and patent documents are cited throughout the specification in order to describe the state of the art to which this invention pertains. Each of these citations is incorporated by reference herein as though set forth in full.

The application of bone cement to bone during surgical procedures, such as the attachment of a prosthesis or pathological fracture fixation, is well known in the surgical community. With regard to the attachment of a prosthesis, the cement is packed into the bone and the prosthesis is then attached. The cement cures and a bond develops between the bone and the prosthesis. Other uses of bone cement include repairing or mending bone fractures or shattered bone occurring from extreme trauma. Bone cement may also be used during cosmetic or dental surgery. Moreover, bone cement may be used as a drug delivery or release system, whereby the bone cement is mixed with antibiotics and applied to a specific surgical site such that the drugs leach out and are delivered directly to the surgical site. Some bone cements are also designed to be absorbed by the body over time.

Typically, the bone cement is prepared by thoroughly blending two components. Bone cement mixtures generally comprise a powdered polymer or copolymer, such as a polymethylmethacrylate (PMMA), and a liquid monomer, usually a methylmethacrylate. Conventionally, the combining of the powder and liquid components is carried out using a container and a spatula or a special mixer resulting in the formation of a quick setting bone cement material. Because of its quick setting nature, the bone cement is usually prepared in the surgical room in conjunction with the surgical procedure. Once the bone cement is thoroughly mixed, the surgeon promptly removes the necessary amount of cement, inserts it into a delivery device or manipulates it by hand, and applies it to the appropriate surface or cavity before the cement mixture cures or hardens.

The incidence of cancer in the United States has risen along with the average lifespan rises and size of population. The American Cancer Society estimates that 1.44 million new cancer cases will be diagnosed in the United States in 2008 (American Cancer Society. Cancer Facts & Figures 2008. Atlanta: American Cancer Society, 2008). This is in addition to the approximately 10.8 million Americans living (2004 estimates) with a history of cancer. As many as half these patients will develop skeletal metastasis, particularly spine metastasis. Spine metastases are generally considered a pre-terminal event, i.e., the cancer is incurable after this stage. Most treatment options, therefore, focus on pain management. Primary tumors of the spine are rare and most patients with spine metastases have primary tumors of the breast, prostate, lung or kidney. Skeletal metastasis may cause increased amount of bone resorption leading to bone instability and fractures (Roodman, G. D. (2004) N. Engl. J. Med., 350:1655-1664). The pathologic fractures caused by these malignant lesions often result in excruciating pain, spinal instability and spinal cord injury. Spinal cord injury associated with pathologic fractures has also been shown to decrease survival.

The treatment options available to prevent fractures include palliative radiation therapy, systemic chemotherapy, and osteoclast inhibiting drugs. These treatment options may also offer pain relief in some patients. However, none of these treatment options address the stability and pain issues caused by severe osteolysis or fracture and consequently, the patient usually needs surgery. A primary objective of surgery is to restore spinal structural integrity. Traditional surgical techniques include removal of the affected bone (e.g., vertebrectomy, laminectomy) and stabilization with various implants (e.g., titanium cages, plates, and bone screws). Many cancer patients are poor candidates for such large, open surgeries. Minimally invasive procedures that use bone cements (e.g., vertebroplasty, kyphoplasty) provide a better alternative treatment option to treat pathologic vertebral fractures in cancer patients.

As stated above, bone cement (e.g., PMMA) may be used to augment weakened bone or bone fractures/defects caused by cancer lesions. To avoid local recurrences of tumor, systemic chemotherapy or radiation therapy may also be administered. In the case of chemotherapy, local drug delivery strategies need to be developed to overcome the unnecessary side effects associated with systemic chemotherapy or radiation treatment. It is an object of the invention to provide improved means for locally treating and preventing tumor recurrence in bone.

### SUMMARY OF THE INVENTION

In accordance with the present invention, an improved drug delivery composition is provided for the treatment of fracture or bone lesions associated with metastasis. An exemplary composition comprises a) at least one suitable biological (biocompatible) polymer; b) at least one soluble filler; and c) at least one therapeutically active agent. Suitable polymers include, without limitation, PMMA and other acrylate polymers. Suitable soluble fillers include glucose, fructose, sucrose, xylitol, glycine, fructose, sodium chloride, sodium carbonate, other soluble salts, and water soluble or biodegradable polymers. Therapeutically active agents to be delivered include at least one chemotherapeutic agent or anti-cancer antibiotic.

In another embodiment of the invention, a method for the treatment of bone fracture or bone lesions associated with metastasis is provided which comprises administration of an effective amount of the drug delivery composition described above to a patient in need thereof.

### BRIEF DESCRIPTION OF THE DRAWINGS

FIG. 1 is a graph showing the effects of various fillers on the elution profile of methotrexate. Lines 1 and 2 are formulations with 25% and 50% glucose, respectively. Lines 3 and 4 are formulations with 25% and 50% NaCl, respectively. Line 5 is a formulation with no filler.

FIG. 2 is a graph showing that addition of glucose to bone cement significantly increases the elution of adriamycin therefrom. Numbers provided are the formula numbers presented in Table 1.

FIG. 3 is graph demonstrating the drug elution properties of Vertebroplastic™ bone cement and Confidence™ bone cement.

FIG. 4 is a graph showing the drug elution from bone cement with no filler, PEG powder filler (particle), and PEG/PVP fiber filler (Nano).

FIG. 5 is a graph showing that increasing PEG concentration in bone cement increases the elution of methotrexate.

FIG. 6 is graph demonstrating the elution of tetracycline from various bone cements.

### DETAILED DESCRIPTION OF THE INVENTION

Metastatic carcinoma is a significant health issue in the United States with over one million cases per year. The skeleton is the third most common site of metastases behind the lung and liver and approximately 500,000 new cases of bone metastases are diagnosed each year. The most common site of bony metastases is the axial skeleton (spine) and significant morbidity may be incurred from the resultant pain, pathologic fractures and even neurologic compromise (Krishnaney et al. (2004) Neurosurg. Clin. N. Am., 15:375-80). In most cases of skeletal metastases, bone osteolysis occurs and an appropriate spacer (e.g., bone cement) is required to restore bone strength and stability. In addition, palliative radiation therapy or chemotherapy is required to prevent recurrence or spread of metastatic lesions. Not all metastases are radiosensitive and systemic chemotherapy may reach toxic levels before it is effective. It is an object of the invention to provide compositions and methods which eliminate the need for radiation or toxic doses of chemotherapy.

Bone cement (e.g., PMMA) can be used as a vehicle to deliver chemotherapeutic drugs locally. This can be done by mixing the desired chemotherapy drug with bone cement before implantation. The combination of bone cement fixation for pathologically weak or fractured bone and local chemotherapy to reduce tumor burden and bone destruction provide a synergistic treatment modality for metastatic bone lesions. In addition, some skeletal metastatic lesions may require very high doses of chemotherapy and mixing chemotherapy drugs to bone cement may help achieve a high localized drug concentration without causing systemic side effects.

Percutaneous vertebroplasty or kyphoplasty is a minimally invasive procedure used to stabilize spine fractures. In these procedures, bone cement (PMMA) may be injected into a bone lesion or fracture through a needle and under fluoroscopic guidance. Once inside the bone, the cement hardens to provide the necessary support and immediate pain relief (Cotten et al. (1999) Radiographics, 19:647-653; Cotten et al. (1996) Radiology, 200:525-530; Lane et al. (2004) Clin. Orthop. Relat. Res., 426:49-53). The exact mechanism by which PMMA bone cement provides immediate pain relief, however, is still unknown. In case of kyphoplasty, inflatable bone tamps (balloon) are first used to restore bone height and a low viscosity bone cement is injected into the preformed void. The creation of preformed voids using bone tamps minimizes the chance of low viscosity cement flowing into the surrounding tissues or vasculature. Bone cement leakage during vertebroplasty procedure can also be minimized by using high viscosity bone cement. Although percutaneous vertebroplasty or kyphoplasty are indicated for the treatment of spine lesions or fractures, similar bone cement based techniques can be used in other skeletal sites.

It should be noted that due to the minimally invasive nature of these procedures, the metastatic lesions are not removed from the bone or adjacent tissues. Additional treatment is needed to prevent metastatic spread in bone as well as the adjacent soft tissues. PMMA itself exhibits anti-tumoral activity in the vicinity of the PMMA implant up to a distance of 5-10 mm (Ruiz et al. (1999) Bone 25:85 S-90S). This can be attributed to thermal necrosis and direct toxicity of the PMMA implant. This anti-tumoral activity is not complete, however, and most of the metastatic tumors extend beyond 10 mm from the implant. Neo-adjuvant radiation or systemic chemotherapy may still be needed to prevent local recurrence or metastatic disease progression.

Systemic chemotherapy often fails to achieve the drug concentration that is needed to destroy or prevent the spread of metastasis after vertebroplasty or kyphoplasty. Additionally, the side effects of chemotherapy and radiation therapy may not be tolerated by these patients. Accordingly, there is a need to develop alternative treatment strategies with minimal side effects. It is worth noting that recently, minimally invasive procedures such as stereotactic radiosurgery or radiofrequency ablation has been used in combination with kyphoplasty to treat metastatic spinal fractures (Halpin et al. (2004) J. Support Oncol., 2:339-355; Singh et al. (2006) J. Bone Joint Surg.-Brit., 88B:434-442). These techniques are expensive, require special training and may not be available in all hospitals. Delivery of drugs locally to bone using PMMA is an attractive option as it may limit the side effects of systemic chemotherapy while at the same time destroying the metastatic lesion and creating stability in the tumor-affected bone. The benefits of local drug therapy include the possibility of achieving high local drug concentration with low systemic toxicity, as well as circumventing local tissue ischemia that might hinder systemic delivery.

There are many types of bone cements (e.g. PMMA or calcium phosphate bone cements) currently available and PMMA based bone cements are currently approved for spine fracture treatment in the United States. There is a concern that during implantation in spine, calcium phosphate based bone cements may cause a fatal pulmonary embolism. PMMA can be used to serve two functions at the same time. In addition to its primary use as a fracture fixation device; PMMA can also function as a drug delivery system. Bone cement's use as a local delivery system for antibiotics and chemotherapeutic drugs has been well documented since 1970 (Bayston et al. J. Bone Joint Surg Br. (1982) 64:460-464; Hernigou, et al. (1989) J. Bone Joint Surg. Br., 71:804-11; Katagiri et al. (1997) Arch. Orthopaed. Trauma Surg., 116:329-333; Futani et al. (2002) J. Orthoped. Sci., 7:262-266; Froschle et al. (1997) Anticancer Res., 17:995-1002; Wang, H. M., et al. (1995) Clin. Orthop. Relat. Res., 312:173-86; Mantha et al. (2005) J. Neurosurg.-Spine, 2:303-307; Fish et al. (1992) Am. J. Hosp. Pharm., 49:2469-2474; Maccauro et al. (2007) J. Mater. Sci.-Mater. Med., 18:839-844; Jiranek et al. (2006) J. Bone Joint Surg. Am., 88:2487-2500; Marks et al. (1976) J. Bone Joint Surg. Am., 58:358-364; Krause et al. (1989) J. Bioactive Compatible Polymers, 4: 345-361; Kim et al. (2001) Intl. Orthoped., 25:343-348). While others have attempted to deliver chemotherapeutic agents (e.g., methotrexate, adriamycin, pirarubicin, and daunorubicin) using PMMA and other bone cement compositions, it is clear that the majority of the drug remained trapped in the cement mixture and thus, ineffective doses were delivered to the tumor (Froschle et al. (1997) Anticancer Res., 17:995-1002; Hernigou et al. (1989) J. Bone Joint Surg. Br., 71:804-811; Katagiri et al. (1997) Arch. Orthoped. Trauma Surg., 116:329-333; Futani et al. (2002) J. Orthoped. Scie., 7:262-266; Wasserlauf et al. (1993) Bull. Hosp. Jt. Dis., 53:68-74; Handal et al. (2007) Clin. Orthopaed. Rel. Res., 459:105-109; Wang et al. (1995) Clin. Orthop. Relat. Res., 312:173-186; Wang et al. (1996) J. Bone Joint Surg. Br., 78:14-17). Antibiotic-loaded PMMA bone cements are regularly used during procedures such as arthroplasty and antibiotic-loaded PMMA bone cement beads are sometimes implanted in soft tissues as a drug delivery system to treat osteomylities. In addition, the feasibility of administering local anesthetics (e.g., prilocaine, bupivacaine, lidocaine) using bone cements has been explored (Wu et al. (2006) Biomaterials, 27:2450-2467). Furthermore, while other materials have been explored for use as a drug delivery depot, PMMA has been the most studied (Healey et al. (2003) Clin. Orthop. Relat. Res., 415 Suppl:S263-275). In summary, PMMA is currently well established in orthopedic literature as an effective stabilizer of fractures and implants through its biomechanical properties. Its combination of strength, biocompatibility and ability to reconstruct bony defects, together with its broad availability, low cost and familiarity to orthopedic surgeons makes PMMA the most appropriate bone drug delivery system currently available.

Drug elution from a non-degradable material such as PMMA depends on a number of factors including, initial volume/weight fraction of the drug in bone cement, surface area of pores, and how well the pores are interconnected. Drugs from currently available bone cement are usually released in a bi-phasic manner; an initial burst followed by tail of very low level drug release that sometimes continues for years (Wu et al. (2006) Biomaterials, 27:2450-2467). Previous research has shown that chemotherapeutic drug elution from bone cement is highest on the first day of implantation and the elution tapers off (e.g., by more than 10-fold) to sub-therapeutic levels on subsequent days (Handal et al. (2007) Clin. Orthop. Rel. Res., 459:105-109). More than 80% of these drugs remain immobilized in the bone cement for years. Chemotherapeutic bone cements with this type of elution offer no benefit to patient and may even cause more harm. For example, when antibiotic bone cement beads (most of them have sub-therapeutic antibiotic drug release profile for years) are left inside the body for a long period of time it causes drug resistance (Neut et al. (2003) Biomaterials, 24:1829-1831).

One way to improve drug elution to therapeutic levels for a few more days is to increase the initial weight or volume fraction of the drug in the bone cement mixture. This can be done by just mixing a large quantity of chemotherapeutic drug to bone cement. However, mixing large quantities of chemotherapy drugs may become quite expensive and may not be advisable for safety reasons. In the case of accidental total release (“drug dumping”), bone cements with large quantity of potent chemotherapy drug, in fact, may prove fatal for a patient. This will also not solve the problem of drug elution at sub-therapeutic levels that continues for years.

To avoid these issues, chemotherapeutic bone cements should have the following properties: 1) bone cements should elute the desired drug at therapeutic and tolerable levels over a period of time (e.g., for at least several days); 2) very little or no drug should remain in the bone cement after a relatively short period of time (e.g., 1-2 months); 3) the initial volume fraction of the drug in bone cement preferably does not greatly exceed a safe dosage limit or at least the initial volume fraction of the chemotherapy drug should be as minimal as possible (e.g., does not exceed a few tens of milligrams); and 4) PMMA bone cement fixations are intended to be permanent and, therefore, the bone cement should be able to provide the necessary support even after complete drug elution. Preferably, the biomechanical properties of the bone cement should match the properties of the surrounding normal bone.

As indicated above, the drug release from currently available bone cements does not follow the ideal zero-order release profile. Indeed, drug release from currently available bone cements is a surface phenomenon and the bulk of the drug remains in the bone cement largely untouched. The release profile from these bone cements can be effective if the released drug concentration at the local tissue level is maintained between the maximum safe concentration and the minimum effective concentration. Currently available bone cements do not have such a release profile.

In stark contrast to the bone cements described above, the bone cements of the instant invention elute the majority, if not all, of therapeutic agent (e.g., chemotherapeutic agent) contained therein to local tissue, optionally at supra-therapeutic concentrations. The bone cements of the instant invention combine the use of soluble fillers with electrospinning of bone cement materials in order to produce a bone cement with unexpectedly superior elution profiles. In addition, the bone cement of the instant invention has reduced mechanical strength due to the porous or fibrous structure. Such reduced mechanical strength may be desirable for spine or other cancellous bone reconstruction applications. Indeed, the reduced mechanical strength is preferred when the bone is weakened by cancer, by natural aging, or by any other factor.

In a particular embodiment of the instant invention, compositions for the production of bone cement are provided. The compositions may be contained within a kit. In a particular embodiment, the bone cement compositions comprise at least one solid composition and at least one liquid composition. In a particular embodiment, the solid composition of the instant invention comprises: 1) at least one soluble filler, 2) at least one therapeutic agent, and 3) at least one bone cement polymer or copolymer. Preferably, the solid composition comprises electrospun material (e.g., the solid is a fibrous mat/contains nanofibers). Preferably, at least one, at least two, or all of the components are electrospun. A layering technique may be used to produce the fibrous mat by electrospinning. The bone cement fibrous mat may be used as such or crushed into smaller pieces of fibrous mats for easy handling and packaging. The liquid composition of the instant invention comprises the monomer of the bone cement polymer or copolymer of the solid composition. In a particular embodiment, the liquid composition does not contain 4-methoxyphenol. The liquid and solid compositions are mixed to form a final bone cement which can be molded and shaped and/or administered to a subject as needed.

Bone cement mixtures generally comprise a powdered polymer or copolymer, such as a polymethylmethacrylate (PMMA), and a liquid monomer, usually a methylmethacrylate. Bone cement is commercially available from a variety of different suppliers, including Depuy (Warsaw, Ind.), Zimmer (Warsaw, Ind.), Orthovita (Malvern, Pa.), Stryker (Kalamazoo, Mich.), Kyphon (Sunnyvale, Calif.) and may be prepared according to manufacturer instructions. Commercially available bone cement may be modified and used in the instant invention. Bone cement polymers of the instant invention include, without limitation, PMMA, PMMA with high molecular weight PMMA (e.g., Confidence™ bone cement), PMMA with styrene, PMMA with polyethylene beads, and glass-ceramic-reinforced Bis-GMA (bisphenol-A-glycidyl dimethacrylate)/Bis-EMA (bisphenol-A-ethoxy dimethacrylate)/TEGDMA (Triethylene glycol dimethacrylate) matrix composite (e.g., Cortoss®).

In a particular embodiment of the instant invention, the therapeutic agent can be incorporated into the bone cement using at least one of the following three methods: a) the therapeutic agent may be in the electrospinning polymer and spun as a fiber, b) the therapeutic agent may be mixed with the polymer powder, and c) the therapeutic agent may be mixed with the liquid monomer. Settling during shelf life should be kept in mind when mixing the therapeutic agent with the liquid monomer. For example, a drug/polymer powder mixture may be prepared by adding methotrexate (e.g., 10-200 mg) or adriamycin (e.g., 1-10 mg) to PMMA (e.g., 2.5 g of powder). The methotrexate should be ground using a mortar or other mechanical device to a fine powder to eliminate clumps and then mixed with the PMMA powder (e.g., by using a spatula). As yet another example of the preparation of a drug/polymer powder mixture, methotrexate or adriamycin may be added to powder PMMA along with water soluble biocompatible polymer powder (e.g., 100-1250 mg).

Most chemotherapeutic agents are potent and effective at very low dosage. In fact, administration of a few milligrams of these drugs may effectively treat certain tumors. For example, adriamycin is a very potent chemotherapy drug used to treat many types of cancers including cancers of the breast, ovarian, bladder and osteogenic sarcomas. One vial of adriamycin typically contains 10 mg of adriamycin and 50 mg stabilizer. When one or two vials of adriamycin are added to a packet of PMMA bone cement (e.g., 22.5 g of powder+9 ml liquid monomer), there is negligible elution from the resultant bone cement composite.

To improve drug elution, the instant invention uses electrospun material and adds soluble space fillers to bone cements. After implantation, the soluble fillers dissolve over time and make the bone cement more porous and interconnected. This helps maintain drug elution at therapeutic levels over a long period of time. Soluble space fillers (e.g. glycine, sucrose, xylitol, erythritol) have been used in bone cements (McLaren et al. (2007) Clin. Orthop. Rel. Res., 461:60-63; McLaren et al. (2007) Clin. Orthop. Rel. Res., 461:64-67; McLaren et al. (2004) Clin. Orthop. Rel. Res., 427:25-27; McLaren et al. (2008) Clin. Orthop. Rel. Res., 466:1372-1376). Dextran has also been used as a filler (Kuechle et al. (1991) Clin. Orthop. Relat. Res., 264:302-308). Lactose and hydroxypropyl methylcellulose (HPMC) have also been used and although HPMC increased the cement porosity, it did not produce an increase in the drug release as HPMC formed a gelatinous capsule around the bone cement specimen (Virto et al. (2003) Biomaterials, 24:79-87). Hydrogen peroxide (foaming agent) and other mechanical methods (drilling) have been used to improve drug elution, although it should be noted that hydrogen peroxide may react with certain therapeutic agents (Shiramizu et al. (2008) J. Orthop. Trauma. 9:17-22). Aqueous sodium hyaluronate solution (Boger et al. (2008) J. Biomed. Mater. Res. Part B, 868:474-482) and carboxymethyl cellulose hydrogels (Bruens et al. (2003) J. Craniofacial Surg., 14:63-68) have also been used before to prepare porous PMMA.

When selecting soluble fillers for chemotherapeutic bone cement applications, the fillers are preferably easily soluble, chemically inert, biocompatible, easily eliminated from the blood stream, and should not cause any adverse reaction to the local tissue at high concentrations. The fillers should also not interfere with the polymerization reaction of bone cement. Studies have shown that adding soluble filler may increase the polymerization reaction time or time to hardening (e.g., by a few minutes). This can be optimized by slightly modifying the polymerization chemistry of these bone cements. Studies have also reported that adding soluble fillers decreases the polymerization temperatures. This may be advantageous as low polymerization temperatures will prevent thermal necrosis of the surrounding tissues (Belkoff et al. (2003) Spine 28:1555-1559). In addition, decreased polymerization temperatures also allow for the use of heat sensitive molecules like peptides or hormones to the bone cement (Hancock et al. (1999) Antimicrob. Agents Chemother., 43:1317-1323; Gelber et al. (2001) Cancer, 92:2172-2180).

Fillers of the instant invention include, without limitation, water soluble biocompatible polymers. In a particular embodiment, the biocompatible filler can be added in the range of 1-20, 1-50, 1-65, 25-50, or 40-60 weight percent. Fillers for use in the formulations of the invention include, without limitation, sugars, polysaccharides, sucrose, dextrose, dextran, glucose, fructose, xylitol, erythritol, glycine, lactose, lactose monohydrate, carboxy methyl cellulose, sodium chloride, poly ethylene glycol (PEG) of different molecular weights, hyaluronic acid of different molecular weights, hydroxyapatite, calcium chloride, calcium sulfate, calcium carbonate, polyvinyl pyrrolidone (PVP), mesoporous silica (Salonen et al. (J. Pharm. Sci. (2008) 97:632-653), sodium alginate, chitosan, hydroxypropylmethylcellulose, amino acids, peptides, N-(2-hydroxypropyl) methacrylamide (HPMA), poly vinyl alcohol, poly anhydrides, and phospholipids. In a particular embodiment, the filler does not interfere with bone cement polymerization (e.g., the filler is not mannitol) and dissolve over time. In a particular embodiment, the filler possesses anti-adhesion properties (e.g., Ofek et al. (FEMS Immunol. Med. Microb. (2003) 38:181-191). Examples of such fillers include, without limitation, lectins, glycoproteins, dendrimers, mannose (e.g., D-mannose), galactose (e.g., D-galactose), and rhamnose (e.g., L-rhamnose). In a particular embodiment, the fillers is selected from the group consisting of sucrose, dextrose, glucose, fructose, xylitol, erythritol, glycine, lactose monohydrate, carboxy methyl cellulose, sodium chloride, PEG, and calcium chloride. In another embodiment, the filler is PEG. In yet another embodiment, the filler is glucose.

The solid compositions of certain bone cements comprise barium sulfate. In a particular embodiment of the instant invention, the barium sulfate is replaced with a metal mesh and/or microfibers. Indeed, a metal mesh may be preferred to barium sulfate because the increased porosity of the bone cement of the instant invention may elute undesirable amounts of barium sulfate to the patient/subject. Metal microfilaments include, without limitation, titanium, stainless steel, tantalum, and magnesium alloy.

Therapeutic agents of the instant invention include, without limitation, chemotherapeutic agents, growth factors, statins (e.g., HMG-CoA reductase inhibitors, atorvastatin (e,g., LIPITOR®), fluvastatin (e.g., LESCOL®, CANEF®), lovastatin (e.g., MEVACOR®), mevastatin, pitavastatin, pravastatin (e.g., PRAVACHOL® or SELEKTINE®), rosuvastatin (e.g., CRESTOR®), and simvastatin (e.g., ZOCOR®), calcilytics, calcimimetics (e.g., cinacalcet, Regpara®, Sensipar®, Mimpara®), anti-inflammatory agents, antibiotics, anesthetics, analgesics, bone growth enhancing agents, sodium bicarbonate, sodium carbonate, peptide drugs (e.g., dhvar-5 (an antimicrobial peptide based on histatin-5), and agents which attract stem cells to the bone metastasis site. A variety of different types of chemotherapeutic agents are contemplated for use herein. These include, without limitation, toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol®)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In a particular embodiment, the chemotherapeutic agent is selected from the group consisting of: placitaxel (Taxon®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives. In a particular embodiment, the bone cement comprises at least one antibiotic. In another embodiment, the bone cement comprises a chemotherapeutic agent and, optionally, at least one statin, sodium carbonate or bicarbonate, or antibiotic.

Antibiotics may also be formulated with bone cement and fillers as disclosed herein. As used herein, the term “antibiotic” refers to antimicrobial agents for use in human therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

The therapeutic agents are used in amounts that are therapeutically effective. While the effective amount of a therapeutic agent will depend on the particular material being used, amounts of the biologically active substance from about 1% to about 65% have been easily incorporated into the present delivery systems while achieving controlled release. Lesser amounts may be used to achieve efficacious levels of treatment for certain therapeutic agents.

As stated hereinabove, bone cement fixations in patients (e.g., spine metastasis patients) may be intended to be permanent. One of the concerns with adding soluble fillers to bone cement is the effect of these fillers on mechanical strength. As the soluble fillers elute it will cause the bone cement to become more porous, thereby decreasing the mechanical strength of bone cement. The Young's modulus of commercially available PMMA bone cements are usually in the range of 2-3 GPa. This is much higher than the modulus of cancellous bone which is in the range of 50-800 MPa. Studies have shown that vertebral augmentation with stiff material such as bone cement increases the risk of fractures in adjacent vertebra (Grados et al. (2000) Rheumatology, 39:1410-1414; Uppin et al. (2003) Radiology, 226:119-124). In the case of cancer patients, one can expect the modulus of cancellous bone to be even in the lower range due to severe osteoporosis. The porous bone cements of the instant invention have lower modulus are therefore better for cancer patients.

The bone cement of the instant invention may be used to deliver at least one therapeutic agent to a subject in need thereof. In a particular embodiment, the bone cement is administered to a bone fracture or break or any other bone loss. In yet another embodiment, the bone cement is used for attaching an artificial implant to a bone. In yet another embodiment, the bone cement is administered to treat cancer (e.g., bone cancer), wherein the bone cement comprises at least one chemotherapeutic agent. Such a method may further comprise radiation therapy or other chemotherapy (e.g., systemic). In a further embodiment of the present invention, a method for administering the bone cement comprises a) mixing the bone cement compositions; b) applying the bone cement mixture to the bone and optionally shaping the bone cement as needed; and c) allowing the bone cement mixture to cure within the bone to form the final cured bone cement.

As stated hereinabove, kits for performing the methods of the instant invention are also provided. In a particular embodiment, the kit comprises at least one solid composition and at least one liquid composition. In a particular embodiment, the solid composition comprises: 1) at least one soluble filler, 2) at least one therapeutic agent, and 3) at least one bone cement polymer or copolymer, wherein the solid composition comprises electrospun fibers. The solid composition may be a fibrous mat or comprise fragments of the fibrous mat (e.g., crushed pieces of fibrous mats for easy handling and packaging). The liquid composition of the instant invention comprises the monomer of the bone cement polymer or copolymer of the solid composition. In a particular embodiment, the liquid composition does not contain 4-methoxyphenol. The liquid and solid compositions are mixed to form a final bone cement which can be molded and shaped and administered to a subject as needed. The kits may further comprise instruction material.

### DEFINITIONS

As used herein, the terms “host,” “subject,” and “patient” refer to any animal, including humans.

A “therapeutically effective amount” of a compound or a pharmaceutical composition refers to an amount effective to prevent, inhibit, or treat the symptoms of a particular disorder or disease. For example, “therapeutically effective amount” may refer to an amount sufficient to inhibit cancer growth.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers may be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (e.g., Remington's Pharmaceutical Sciences, 18th Ed. (1990, Mack Publishing Co., Easton, Pa. 18042)). Pharmaceutically acceptable carriers may be prepared from a wide range of materials. Carriers also include, without limitation, binders, adhesives, lubricants, disintegrants, colorants, bulking agents, and miscellaneous materials such as buffers and adsorbents in order to prepare a particular composition.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain without a loss of consciousness). Analgesics include opioid analgesics (e.g., codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine) and non-opiate analgesics (e.g., NSAIDs such as salicylates (e.g., aspirin, methyl salicylate, and diflunisal); arylalkanoic acids (e.g., indomethacin, sulindac, diclofenac, and tolmetin); N-arylanthranilic acids (e.g., fenamic acids, mefenamic acid, and mecflofenamate); oxicams (e.g., piroxicam and meloxicam); coxibs (e.g., celecoxib, rofecoxib, valdecoxib, parecoxib, and etoricoxib); sulphonanilides (e.g., nimesulide); naphthylalkanones (e.g., nabumetone); anthranilic acids (e.g., pyrazolidinediones and phenylbutazone); proprionic acids (e.g., fenoprofen, flurbiprofen, ibuprofen, ketoprofen, naproxen, and oxaprozin); pyranocarboxylic acids (e.g., etodolac); pyrrolizine carboxylic acids (e.g., ketorolac); and carboxylic acids.

As used, the term “anesthetic” refers to an agent that produces a reversible loss of sensation in an area of a subject's body. An agent may act as both an analgesic and an anesthetic. Anesthetics include, without limitation, benzocaine, benzyl alcohol, bupivacaine, butamben picrate, chlorprocaine, cocaine, dibucaine, dimethisoquin, dyclonine, etidocaine, hexylcaine, ketamine, lidocaine, mepivacaine, phenol, pramoxine, procaine, tetracaine, salicylates, ropivacaine, prilocaine, and xylocaine.

As used herein, the term “calcilytic” generally refers to compounds able to inhibit calcium receptor activity and more specifically refers to compounds that inhibit, block, or decrease calcium sensing receptor (CaSR) activity. A calcilytic may, for example, block, either partially or completely, the ability of increased concentrations of extracellular Ca²⁺ to (a) increase [Ca²⁺]; (b) mobilize intracellular Ca²⁺; (c) increase the formation of inositol-1,4,5-triphosphate; and/or (d) decrease dopamine or isoproterenol-stimulated cyclic AMP formation. Calcilytic compounds include, without limitation, those disclosed in European Patent and Publications Nos. 637,237; 724,561; 901,459; 973,730; 1,258,471; 1,466,888; and 1,509,518; International Publication Nos. WO 97/37967; WO 99/51569; WO 04/017908; WO 04/041755; WO 04/047751; WO 05/030746; and WO 05/030749; U.S. Pat. Nos. 6,395,919; 6,432,656; 6,521,667; 6,750,255; 6,818,660; 6,864,267; 6,908,935; 6,916,956; 7,109,238; 7,202,261; and U.S. Patent Application Publication Nos. 2004/0009980 and 2004/0014723.

As used herein, the term “calcimimetic” refers to a compound that binds to calcium sensing receptors and induces a conformational change that reduces the threshold for calcium sensing receptor activation by the endogenous ligand Ca²⁺. Calcimimetic compounds include, without limitation, those disclosed in European Patent Nos. 637,237; 657,029; 724,561; 787,122; 907,631; 933,354; 1,203,761; 1,235,797; 1,258,471; 1,275,635; 1,281,702; 1,284,963; 1,296,142; 1,308,436; 1,509,497; 1,509,518; and 1,553,078; International Publication Nos. WO 93/04373; WO 94/18959; WO 95/11221; WO 96/12697; WO 97/41090; WO 01/34562; WO 01/90069; PCT/EP2006/004166; WO 02/14259; WO 02/059102; WO 03/099776; WO 03/099814; WO 04/017908; WO 04/094362; WO 04/106280; WO06/117211; and WO 06/123725; U.S. Pat. Nos. 5,688,938; 5,763,569; 5,962,314; 5,981,599; 6,001,884; 6,011,068; 6,031,003; 6,172,091; 6,211,244; 6,313,146; 6,342,532; 6,362,231; 6,432,656; 6,710,088; 6,750,255; 6,908,935; 6,916,956; 6,939,895; 7,084,167; 7,157,498; 7,196,102; 7,211,685; 7,304,174; and 7,176,322; and U.S. Patent Application Publication No. 2003/0176485. A compound may act as both a calcimimetic and a calcilytic.

As used herein, the phrase “bone growth enhancing agent” refers to a compound which increases the rate of bone growth. Bone growth enhancing agents include, without limitation, bone morphogenetic proteins (BMPs), cytokines, hormones, and growth factors.

In this invention, the terms “(meth)acrylate” and “poly(meth)acrylate” include the monomers and polymers, respectively, of methacrylic acid esters and acrylic acid esters, and the polymers also include the co-polymers of the compounds named.

As used herein, the term “electrospinning” refers to the production of fibers (i.e., electrospun fibers), particularly nano-sized fibers, from a solution or melt using interactions between fluid dynamics and charged surfaces (e.g., by streaming a solution or melt through an orifice in response to an electric field). The dried or solidified fibers typically have diameters of about 40 nm, or from about 10 to about 100 nm, although 100 to 500 nm fibers are commonly observed. Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

As used herein, the term “bone cancer” refers to both primary and secondary bone cancers. Primary bone cancer refers to cancers which start in the bone, whereas secondary bone cancers refers to cancers which start in other parts of the body, such as breasts, lung, and prostate, and later metastasize to bone. Bone cancers include, without limitation, osteosarcomas, chondrosarcomas, and osteocarcinomas.

The following examples are provided to illustrate certain embodiments of the invention. They are not intended to limit the invention in any way.

### Example 1

When an antibiotic or chemotherapy drug is added to PMMA and tested in vitro, there is a surge in drug elution for the first 24 hours. Elution then tapers off to a level which is often sub-therapeutic. The majority of the drug (−75%) remains trapped in the cement for a long period of time. Thus, the simple addition of chemotherapeutic drugs alone to a PMMA formulation is not sufficient for localized drug delivery at the site of the lesion for a therapeutically relevant time period.

Drug elution from PMMA depends on a number of factors including, drug to PMMA ratio, surface area and nature of interconnected pores. One way to improve drug elution is to increase the drug to PMMA ratio. This can be done by mixing a large quantity of chemotherapeutic drug with the PMMA. However, most chemotherapy drugs cannot be tolerated at high doses and a very small quantity is usually administered during systemic chemotherapy. This caps the maximum amount of chemotherapy drug that can be added to PMMA because if all of the drug were to be eluted in a 24 hour period, the patient may experience an adverse toxicity event. Additionally, very high drug concentrations can interfere with polymerization of the bone cement. Therefore, this approach may not be feasible for safety reasons. The other options include compositions and methods which increase surface area, porosity and more importantly interconnectivity of pores.

Surface area, porosity and interconnectivity of pores can be improved by addition of soluble fillers, which include, but are not limited to sucrose, dextrose, glucose, fructose, xylitol, erythritol, glycine, lactose monohydrate, carboxy methyl cellulose, sodium chloride, poly ethylene glycol, and calcium chloride that dissolve over time. For example, common salt, glucose or fructose can be mixed with bone cement and chemotherapy drugs. With time, the soluble fillers will dissolve and make the bone cement more porous and interconnected. This facilitates maintenance of drug elution at therapeutic levels for a greater duration than previously reported.

Table 1 provides different formulations of bone cement, filler and chemotherapeutic drug which were tested for effects on increasing or maintaining elution profiles.

TABLE 1

| | Formula | Formula | Formula | Formula | Formula |
|---|---|---|---|---|---|
| | 1 | 2 | 3 | 4 | 5 |
| Composition | | | | | |
| Powder (g) | 2.5 | 2 | 1.75 | 1.5 | 1.25 |
| Monomer (ml) | 1 | 1 | 1 | 1 | 1 |
| Sugar/salt (g) | 0 | 0.5 | 0.75 | 1 | 1.25 |
| Drug | | | | | |
| Methotrexate | 105 mg | 105 mg | 105 mg | 105 mg | 105 mg |
| Adriyamycin | 6 mg | 6 mg | 6 mg | 6 mg | 6 mg |
| Carboplatin | 50 mg | 50 mg | 50 mg | 50 mg | 50 mg |

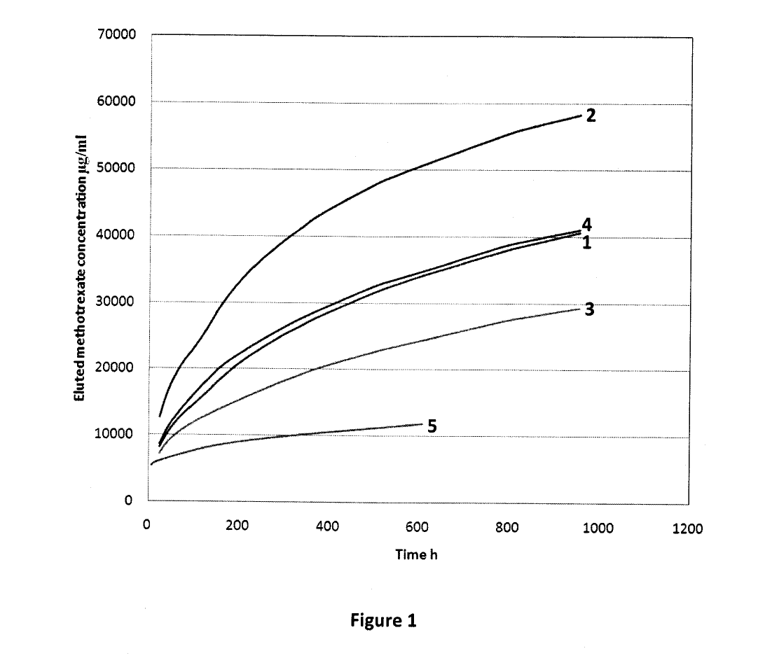

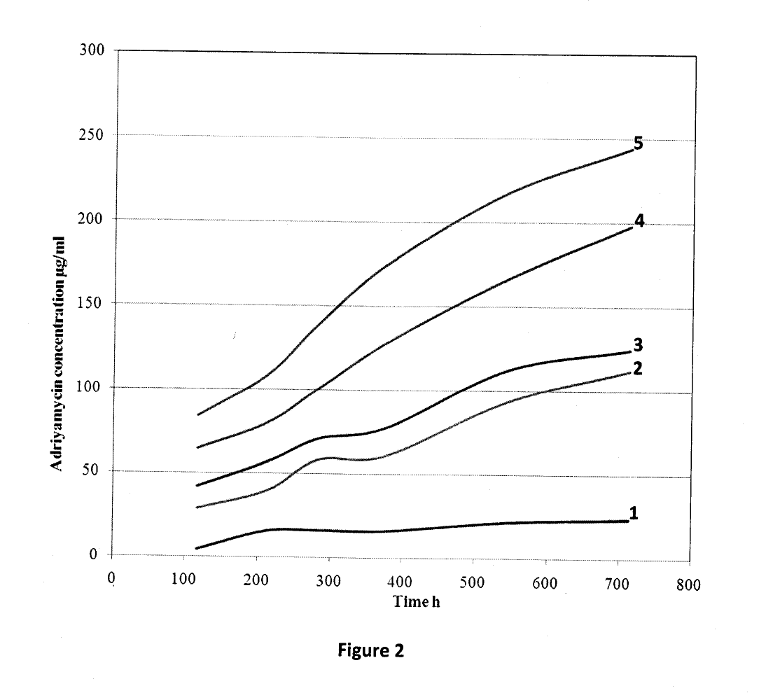

The elution results using the formulations provided above are shown in FIG. 1. The data show that addition of fillers to the bone cement/chemotherapeutic drug combination significantly prolongs elution of the drug. FIG. 2 shows a similar result when the elution of adriamycin was tested in the presence of different glucose filler weight percentages. The weight percentages are in relation to the amount of solid PMMA (not the liquid monomer).

Thus, the present inventors have discovered a three component compositions and methods of use thereof for the treatment of fracture and bone lesions due to metastatic cancer.

### Example 2

Drugs from bone cement are usually released in a bi-phasic manner, namely, an initial burst followed by a tail of low level drug release that continues for years. This is not ideal in for both antibiotics and chemotherapy drugs. Drug elution can be improved by adding soluble fillers or porogens that increase pore interconnectivity. Soluble fillers reported in antibiotic bone cement literature include PVP, glycine, dextran, xylitol, lactose, dhvar-5, chitosan and hydroxypropylmethylcellulose. However, some fillers interfere with bone cement polymerization (e.g. mannitol).

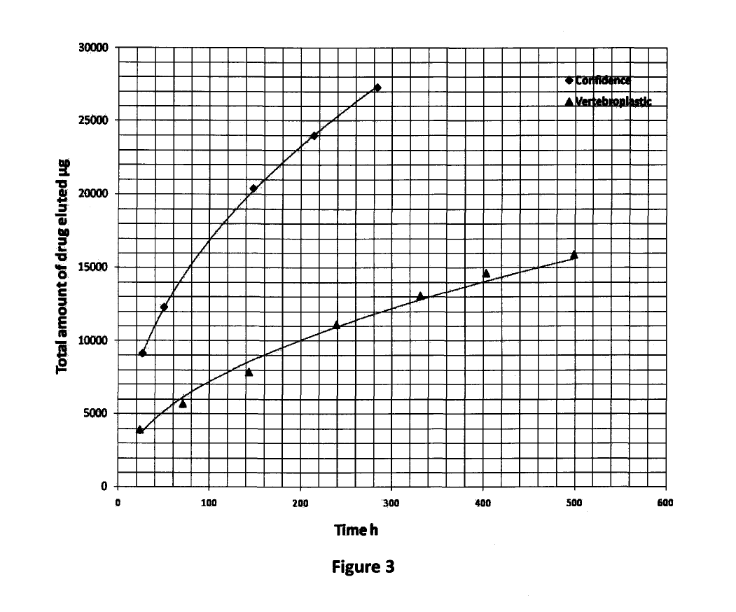

Methotrexate release can be altered by changing the bone cement components. The formula for Vertebroplastic™ bone cement is:
Powder
Methylmethacrylate polymer 56.8% w/w
Methylmethacrylate-styrene copolymer 14.2 w/w
Benzoyl peroxide 0.4% w/w
Barium sulfate 28.6% w/w
Liquid
Methylmethacrylate monomer 95.05% v/v
Ethylene dimethacrylate monomer 4.28% v/v
Dimethyl-p-toluidine 0.67% v/v
Hydroquinone 20±5 ppm
4-methoxyphenol 12 ppm
   By changing the liquid monomer (e.g., removing 4-methoxyphenol and ethylene dimethacrylate monomer), the elution of methotrexate is improved (FIG. 3). The modified bone cement comprises:
Powder
Methylmethacrylate polymer 56.8% w/w
Methylmethacrylate-styrene copolymer 14.2 w/w
Benzoyl peroxide 0.4% w/w
Barium sulfate 28.6% w/w
Liquid (used with Confidence™ cement)
Methylmethacrylate monomer 98.5% v/v
N,N-dimethyl-p-toluidine 1.5% v/v
Hydroquinone 20 ppm

### Example 3

Methotrexate release can be altered by using nano/microfibers. The soluble fillers can be spun into polymer nanofibers.

The preparation of the chemotherapeutic bone cement/fiber composite comprises:

Step 1: Mixing bone cement powder (Vertebroplastic™, 2.5 g) with Methotrexate (100 mg)

Step 2: Loading it in a manual or air powered dispenser

Step 3: Preparing 1 cc polymer solution (e.g. 4 g PVP+3 g Polyethylene glycol)

Step 4: Electrospinning a thin layer of polymer solution for 15-30 sec

Step 5: Dispensing a thin layer of bone cement powder

Step 6: Repeating step 4 and step 5

Step 7: Drying the fibrous bone cement mat for 2 hours at 37° C. and then in vacuum chamber for 1 hour.

More specifically, the electrospinning of the polymer solution composition comprises the following. 4 g polyethylene glycol (PEG-MW 8000) and 3 g polyvinyl pyrrolidone (PVP) were added to 40 ml of ethanol and 5 ml of distilled water to form the electrospinning polymer solution. The solution was stirred for 30 minutes and ultrasonicated in the degas mode for another 10 minutes to remove any air bubbles. The prepared polymer solution was electrospun using a standard electrospinning setup that included an automated syringe pump, a high voltage source, and a grounded aluminum collector plate. The electrospinning voltage can be anywhere between 8-25 KV. A voltage of 20 KV was used in this experiment. The distance between the electrospinning nozzle and the collector plate can be anywhere between 10-20 cm. 1 ml of this polymer solution yields 150 mg of nano or micro fibers after drying. Electrospun fibers can be mixed directly with bone cement mixtures; however they clump together and cannot be mixed uniformly. In addition, the nanostructure may be lost in the mixing process. This can be avoided by using a layering technique. A desired drug can also be added to the electrospinning polymer solution.

The modified bone cement comprises

Powder (2.5 g)

Methylmethacrylate polymer 56.8% w/w

Methylmethacrylate-styrene copolymer 14.2 w/w

Benzoyl peroxide 0.4% w/w

Barium sulfate 28.6% w/w

Liquid (used with confidence cement) 1.2 ml

Methylmethacrylate monomer 98.5% v/v

N,N-dimethyl-p-toluidine 1.5% v/v

Hydroquinone 20 ppm

100 mg Methotrexate

150 mg Nanofiber

The prepared bone cement fibrous mat (2.6 g) was then mixed with 1.2 ml of methylmethacrylate monomer and molded into a cylindrical elution test specimen. It should be noted that the monomer components play a major role in the rate of elution. Liquid monomer containing methylmethacrylate monomer 98.5% v/v, N,N-dimethyl-p-toluidine 1.5% v/v and Hydroquinone 20 ppm was used in this experiment. Glass vials were used as molds to prepare the cylindrical specimens for elution studies. The polymerized cylindrical bone cement specimens were then taken out of the glass molds by breaking the glass molds carefully.

The cylindrical bone cement specimens were placed in an air tight plastic vial containing 20 ml saline (elution media). The vials were placed in an incubator maintained at 37° C. Methotrexate elution was measured at different time points for 670 hours. The eluted methotrexate concentration was measured in triplicates using a micro-plate reader (Spectramax® 190, Molecular Devices, CA) at a wavelength of 405 nm. The elution media was replaced whenever the methotrexate concentration was measured. The amount of methotrexate eluted was plotted as a function of time.

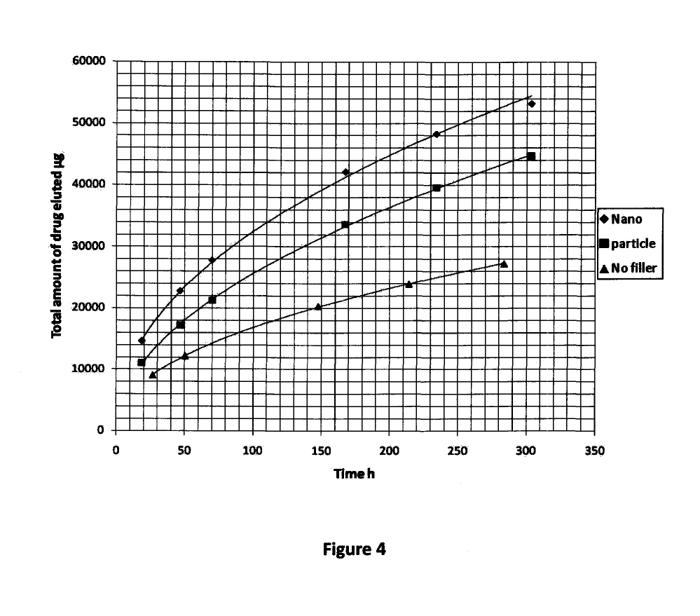

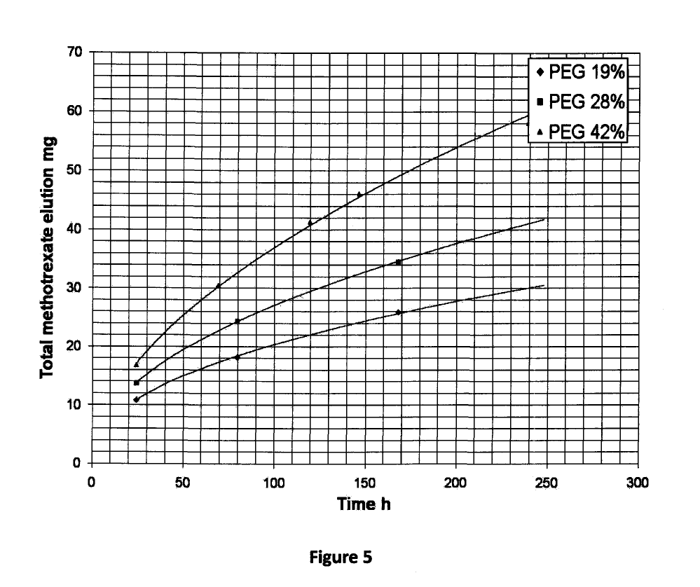

FIG. 4 provides a comparison of elution with no filler, PEG powder (150 mg) and PEG/PVP fiber (150 mg). As seen in FIG. 4, the combination of soluble filler (PEG) with nanofibers leads to greater elution of the chemotherapeutic drug consistently over time. FIG. 5 shows that increasing the amount of 3350 MW poly ethylene glycol powder increased elution of methotrexate (initially 100 mg).

### Example 4

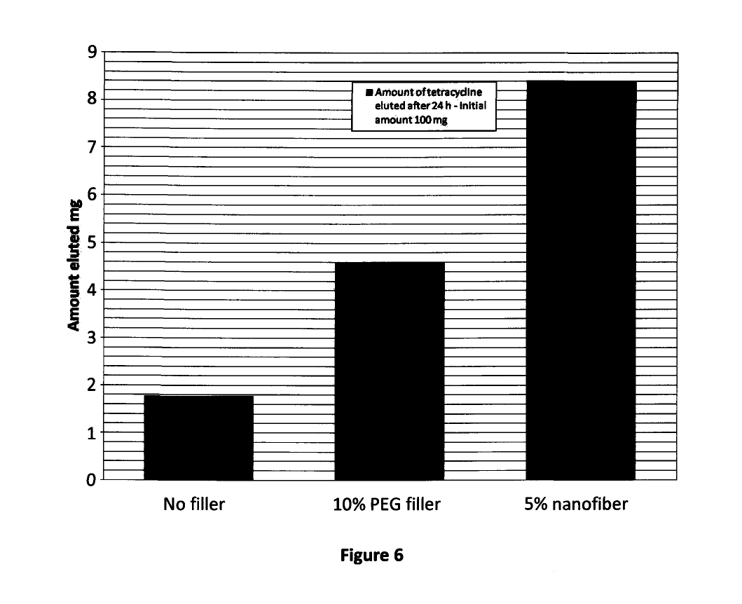

Three different bone cement specimens were prepared: 1) 2.5 g of Vertebroplastic™ cement was mixed with 100 mg tetracycline hydrochloride; 2) 2 g of Vertebroplastic™ cement was mixed with 0.5 g of poly ethylene glycol (mw 3350) and 100 mg tetracycline hydrochloride; and 3) 2.5 g of Vertebroplastic™ cement was mixed with 100 mg tetracycline hydrochloride and 1 ml of PVP/PEG solution containing 150 mg PVP/PEG was electrospun in a layered fashion on to the prepared Vertebroplastic™/tetracycline mixture. 1 ml Confidence™ monomer was used to polymerize specimens 1 and 2. 1.2 ml Confidence™ monomer was used to polymerize specimen 3. FIG. 6 demonstrates that electrospinning allows for greater elution of the therapeutic agent.

### Example 5

For bone cement composite preparation, 8 g of Confidence™ ultra bone cement (Depuy Spine Inc., MA) was weighed and mixed with 2 g of poly ethylene glycol powder (3500 molecular weight) in a biologic safety cabin. After thorough mixing, 4 g of this mixture was added to a vial of 10 mg adriamycin. They were mixed thoroughly to ensure there are no large adriamycin flakes. The final bone cement mixture contained 2.5 mg of adriamycin per 1 g bone cement-poly ethylene glycol powder. One gram of this composite elutes 340 micrograms of adriamycin within 24 hours in vitro. The prepared bone cement drug powder was then loaded in sterile syringes (2 g/syringe). To prepare the bone cement putty for implantation and in situ polymerization, 1 ml of Confidence™ monomer was added to this powder and mixed thoroughly 5 minutes prior to implantation.

For the implantation of bone cement/filler/drug in an animal model, two male New Zealand White rabbits (approximate age 6 months; Covance, Pa.) were used in this study to collect preliminary data on whether the bone cement/filler/drug composite caused any immediate adverse systemic toxicity. After anesthesia and pre-operative rabbit preparation, the tibia was exposed though a 1 cm anterior incision and a 15-gauge intraosseous infusion needle or Jamshidi® bone marrow needle was inserted through the proximal tibia into the marrow cavity (monitored using x-ray). The syringe containing the bone cement putty was connected to the needle and 1.5 g of bone cement was injected into the bone marrow cavity. The rabbits were monitored for 7 days and euthanized at the end of 7 days. The lungs and tibias with surrounding soft tissues were removed after euthanasia and fixed in formalin and the tibia will be sectioned with bone and soft tissue for microscopy. The sections were used to determine the presence of emboli and the amount of bone destruction and number of osteoclasts. The histology sections did not reveal the presence of any pulmonary emboli due to bone cement implantation. The cortical bone surrounding the cement implant also did not reveal any abnormal activity nor toxicity.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A bone cement for delivering at least one therapeutic agent to a subject in need thereof, said bone cement comprising:
a) at least one bone cement polymer,
b) at least one soluble filler, and
c) at least one therapeutic agent,
wherein said bone cement comprises electrospun fibers.

2. The bone cement of claim 1, wherein said bone cement polymer is polymethylmethacrylate.

3. The bone cement of claim 1, wherein said filler is a polysaccharide.

4. The bone cement of claim 1, wherein said filler is selected from the group consisting of glucose, sucrose, dextrose, fructose, xylitol, erythritol, glycine, lactose monohydrate, carboxy methyl cellulose, sodium chloride, polyethylene glycol, sodium bicarbonate and calcium chloride.

5. The bone cement of claim 4, wherein said filler is glucose.

6. The bone cement of claim 4, wherein said filler is polyethylene glycol.

7. The bone cement of claim 1, wherein said therapeutic agent is a chemotherapeutic drug.

8. The bone cement of claim 1, wherein said at least one therapeutic agent comprises at least one statin and at least one chemotherapeutic drug.

9. The bone cement of claim 1, wherein said at least one therapeutic agent comprises sodium carbonate and at least one chemotherapeutic drug.

10. The bone cement of claim 1, wherein said therapeutic agent is an antibiotic.

11. The bone cement of claim 7, wherein said bone cement polymer is polymethylmethacrylate, said filler is glucose, and said chemotherapeutic drug is methotrexate or adriyamycin.

12. A method for delivering a therapeutic agent to a subject in need thereof, said method comprising administering the bone cement of claim 1.

13. A method for the treatment of a bone fracture in a subject comprising administration of an effective amount of the bone cement of claim 1 to said subject.

14. A method for the treatment of a bone cancer in a subject comprising administration of an effective amount of the bone cement of claim 7 to said subject.

15. A kit for producing the bone cement of claim 1, said kit comprising:
a) at least one solid composition comprising 1) at least one soluble filler, 2) at least one therapeutic agent, and 3) at least one bone cement polymer or copolymer; and
b) at least one liquid composition comprising the monomer of the bone cement polymer or copolymer of the solid composition.

16. A method for preparing a bone cement, said method comprising:
a) electrospinning at least one component of a solid bone cement composition, wherein said solid bone cement composition comprises at least one bone cement polymer or copolymer, at least one filler, and, optionally, at least one therapeutic agent; and
b) mixing a liquid bone cement composition with the electrospun material of step a), wherein said liquid bone cement composition comprises the monomer of the bone cement polymer or copolymer of the solid composition and, optionally, at least one therapeutic agent.

17. The method of claim 16, wherein said filler is electrospun.

18. The method of claim 16, wherein said solid bone cement composition comprises at least one therapeutic agent.

19. The method of claim 16, wherein step a) comprises layering electrospun filler with electrospun bone cement copolymer.
